(19) United States
(12) Patent Application Publication
(10) Pub. No.: US 2016/0135670 A1
(43) Pub. Date: May 19, 2016

(54) APPARATUS FOR PROVIDING IMAGING SUPPORT DURING A SURGICAL INTERVENTION

(71) Applicant: MAQUET GMBH, Rastatt (DE)

(72) Inventors: Michael Bernhart, Karlsruhe (DE); Bastian Ibach, Karlsruhe (DE); Kathrin Roberts, Karlsruhe (DE); Holger Borchers, Aachen (DE)

(73) Assignee: MAQUET GMBH, Rastatt (DE)

(21) Appl. No.: 15/006,612

(22) Filed: Jan. 26, 2016

Related U.S. Application Data
(63) Continuation-in-part of application No. PCT/EP2014/066523, filed on Jul. 31, 2014.

(30) Foreign Application Priority Data
Jul. 31, 2013 (DE) 10 2013 108 228.9

(51) Int. Cl. A61B 1/045; A61B 1/00; G06T 7/00; H04N 5/225; H04N 5/232

(52) U.S. Cl. CPC A61B 1/045; H04N 5/2257; H04N 5/2251; H04N 5/23293; H04N 5/23296; H04N 5/23216; H04N 5/23245; G06T 7/0044; A61B 1/00149; A61B 1/00045; A61B 1/00066; H04N 2005/2255; A61B 2034/301

(57) ABSTRACT

A surgical operation imaging device is disclosed. The surgical operation imaging device has an endoscope having a camera that generates image data, a manipulator that moves the endoscope, and a viewing device that presents a moving image based on the image data. The surgical operation imaging device also has a controller that controls the manipulator and the endoscope based on a plurality of operating states and an operating element coupled with the controller. The operating element has a first switching state and a second switching state. A selected operating state is selected from the plurality of operating states based on the operating element switching between the first switching state and the second switching state. The selected operating state is changed based on the operating element switching between the first switching state and the second switching state.

### CROSS REFERENCE TO RELATED APPLICATIONS

The present application is a continuation-in-part filed under 35 U.S.C. §111(a), and claims the benefit under 35 U.S.C. §§365(c) and 371 of PCT International Application No. PCT/EP2014/066523, filed Jul. 31, 2014, and which designates the United States of America, and German Patent Application No. 10 2013 108 228.9, filed Jul. 31, 2013. The disclosures of these applications are herein incorporated by reference in their entirety.

### TECHNICAL FIELD

The present disclosure is directed to an assistance device for imaging support of a surgeon during a surgical operation, comprising an endoscope with a camera for generation of image data, a viewing device for presenting a moving image on the basis of the image data generated by the camera, a manipulator coupled with the endoscope for moving the endoscope, and a control unit for optional actuating of the manipulator in accordance with one of several operating states, such that the displayed moving image can be influenced by moving the endoscope in accordance with the particular selected operating state.

### BACKGROUND

During a laparoscopic procedure, a surgeon typically looks at a moving image of an operation site on a viewer, such as a monitor. In the moving image one can see the laparoscopic instrument with which the surgeon is manipulating the anatomical structures and organs in the patient. The moving image presented on the viewer in real time is recorded by a camera, which is typically part of an endoscope introduced into the body of the patient via a trochar and directed to the operation site.

The endoscope is usually held by an assistant, who stands alongside the surgeon during the operation. The assistant usually tries to direct the endoscope at the operation site so that the target region in which the tips of the instruments and the anatomical structures being manipulated can be seen is located in a reference position of the moving image. Usually this reference position lies roughly in the middle of the moving image. If the image section being viewed needs to be changed, the endoscope may be moved in order to bring the new target region again into the middle of the moving image.

Various image movements are relevant to the surgeon looking at the viewer. Typically, there are two-dimensional changes in the image section, e.g., movements of the image section directed upward and downward on the viewer, movements of the image section directed left and right on the viewer, as well as combined movements, such as from bottom left to top right. Moreover, the image section on the viewer may also be changed by a corresponding zoom operation in the third dimension, e.g., enlarging the image within a reduced field of view, and/or reducing the image within an enlarged field of view.

Assistance devices for imaging support of a surgeon without using a human assistant are known from the prior art. Accordingly, these assistance devices are able to be operated by the surgeon himself through corresponding control commands.

For example, an operating concept is known from the prior art in which the surgeon can dictate the direction of movement of the image presented on the viewer by the surgeon's own head movement. Also, for example, a movement of an endoscope movement is enabled by an activation pedal. For this, reference is made for example to the publications EP 2169348 B8, EP 2052675 A1, US 2009/0112056 A1, EP 0761177 B1 and U.S. Pat. No. 5,766,126 A.

Other operating concepts call for a voice control or a control of the endoscope movement via a foot pedal outfitted with several switches. For this, reference is made to the publications U.S. Pat. No. 6,932,089 B1, US 2006/0100501 A1 and US 2011/0257475 A.

Assistance devices which enable an automatic tracking of marked instruments are described in publications U.S. Pat. No. 6,820,545 A1 and DE 19529950 C1.

Publication EP 1937177 B1 proposes, for the operation of an assistance device, an operating lever also known as a joystick, which is arranged on a laparoscopic instrument. The surgeon can thus also control the endoscope movement with his hand holding the instrument.

Finally, a fully automatic system which enables an automatic tracking of marked instruments is described in DE 199 61 971 B4.

### SUMMARY OF THE DISCLOSURE

Despite the above documented multitude of technical solutions, there still exists the desire to optimize an assistance device so that on the one hand it enables the suitable automation of the endoscope tracking, so that the surgeon is not burdened with the operating of the assistance device. Furthermore, the operation of such an assistance device may be easy to learn and easy to carry out.

The present disclosure provides an assistance device which the surgeon can operate easily and intuitively during a surgical operation.

Various embodiments of the present disclosure address the above problem in an assistance device via an operating element or member (e.g., an operating assembly such as a mechanical operating assembly) coupled with the control unit for the selecting of a particular operating state, wherein the operating element may be formed from a single switch element with two switching states, of which one switching state is an activated state and the other switching state is a non-activated state, and wherein mutually distinguishable switching patterns may be coordinated with the operating states on the basis of the two possible switching states of the switch element, which can be generated with the help of the switch element.

By providing a single switch element having two switching states, it is possible for the operator to direct the assistance device in an easy manner where to move the endoscope coupled with the manipulator in order to bring about the desired image presentation on the viewing device. The switch element according to the present disclosure may form, for example, a “one-dimensional” operating element, by which the intraoperative control of the assistance device can be kept relatively simple. On the basis of the two switching states of the switch element, easily learned switching patterns can be predetermined, which the surgeon can generate by a corresponding activation of the switch element in order to set the desired operating state.

The function of the switch element is not confined to the intraoperative control of the assistance device. Thus, the switch element can be used in addition, for example, to perform a setup or adjustment process prior to the actual operation of the assistance device, in which control parameters are set and/or operating states are configured.

Since the surgeon may only operate a single switch element, the handling may be, for example, simplified. Thus, the surgeon can find the switch element at all times without looking at it, so that his concentration on the surgical procedure being performed is not disturbed. The single switch element can be easily and distinctly positioned, for example on the medical instrument with which the surgeon is performing the operation, or on the hand or fingers of the surgeon. This procedure may be unsuitable for an operating unit or surgical theater having more than two operating elements or tools.

Since only a single switch element may be available by which all functions of the assistance device that are used during the operation can be controlled, an operator may not have to learn any coordination of certain functions with different operating elements. For example, substantially all functions may be coordinated (e.g., solely) with this one switch element.

Since a single switch element with two switching states may be sufficient for the realization of the operating element according embodiments of the present disclosure, one can use a foot switch with a single pedal for this (e.g., as is suitable for the control of other technical systems in the operating room). Likewise, it is possible to place a correspondingly small-sized switch or push button on the handle of the instrument used for the operation. For example, a switch element which is sterile and can be triggered without setting aside the medical instrument can be used if it is able to generate the switching patterns according to the present disclosure.

There may be provided, for at least one of the operating states selectable by the switching pattern, a predetermined actuation operation of the control unit for movement of the endoscope specific to the operating state. The control unit after selection of this operating state may then perform the corresponding actuation operation continuously, while the switch element is in the activated state. In this exemplary embodiment, for example, the switch element may be pressed to select a particular operating state and the actuation operation predetermined for this operating state is executed as long as the switch element is held (e.g., held pressed down or otherwise actuated). In this way, the surgeon with a single activating of the switch element can bring about a switching between two operating states and at the same time order the predetermined actuation of the endoscope for the newly selected operating state.

The control unit after selection of the operating state may switch to a ground state (e.g., a default initial state) if the switch element remains in the non-activated state for longer than a predetermined period of time. Thus, the surgeon may be notified that the assistance device may be constantly in a well-defined ground state without activating the switch element, from which the individual operating states can be selected. This may also make it easier (e.g., intuitively easier) for the surgeon to learn the operating concept according to the present disclosure.

The switching patterns may involve at least one single pulselike activation of the switch element and/or at least one activating of the switch element lasting longer than a certain activation time. A single pulselike activation may indicate a one-time triggering of the switch element, which may move and/or transition from the non-activated switching state only briefly to the activated switching state and then back to the non-activated switching state. For example, a pulselike activation may include an activation of the switch element for a relatively short duration of time (such as, for example, a clicking of the switching element, for example, the clicking of a button when, e.g., the switching element is a button or similar mechanical device). With these pulselike activations one can predetermine sequences of trigger signals in a relatively simplified manner, which may be simple to learn. These activations may also be suitable for placing the assistance device in the various operating states. On the other hand, a longer lasting activation of the switch element can be provided, during an operating state that has been selected, to carry out the actuation operation corresponding to this state (e.g., so as to move the endoscope continuously for as long as the activation of the switch element is held in place).

The switching patterns coordinated with at least one portion of the operating states may be configured to build on one another in succession, so that the corresponding operating states can be selected one after another with the aid of the switch element in a predetermined sequence. In this way, the surgeon can move the assistance device successively from one operating state to the next in an easily-learned manner.

The control unit may contain an image processing module, which may detect at least one medical instrument in the displayed moving image on the basis of the image data generated by the camera and may ascertain a position deviation of the medical instrument in regard to (e.g., relative to) a reference position established within the displayed moving image. One of the operating states selectable with the switch element may be a tracking state, in which the control unit determines with the help of the position deviation a nominal value and actuates the manipulator in dependence on this nominal value so that the medical instrument detected in the displayed moving image may be brought into the determined reference position by tracking of the endoscope. The image processing module may detect a tip of the medical instrument and may ascertain the position deviation of this tip. In this embodiment, the instrument recognition carried out by the image processing module and providing information about the position of the instrument tip in the moving image can be combined with the functioning of the switch element according to the invention, serving here as an enabling element. Thus, it is possible to move the moving image dynamically in any desired direction with the instrument tip. For example, the moving image follows the recognized instrument for as long as the switch element acting as an enabling switch is pressed down. Thus, in addition to enabling movements in fixed predetermined directions, such as up or down or left or right, one can realize any desired directions of movement.

The above explained function of the switch element (e.g., in the sense of an enabling switch used in the tracking stat)e can be realized easily when the switching pattern with which the tracking state can be selected includes for example a single change of state, leading from the non-activated state to the activated state of the switch element.

In another exemplary embodiment, the control unit with the change of state from the non-activated state to the activated state of the switch element may start the continuous actuation of the manipulator for the tracking of the endoscope and this actuation may end with the subsequent change of state from the activated state to the non-activated state of the switch element. Thus, for as long as the surgeon holds down the switch element the endoscope may be tracked and the image section displayed on the viewing device will be moved. Once the surgeon releases the switch element, the image section stops and remains stationary.

One of the operating states selectable with the switch element may be an instrument selection state, in which one of several medical instruments which have been detected by the image processing module in the moving image can be selected with the help of the switch element as a guiding instrument, which is brought into the determined reference position in the tracking state by the tracking of the endoscope within the moving image. In this embodiment, one can easily use the switch element to make a change of the guiding instrument used during the tracking of the endoscope.

A graphic user interface may be provided, which displays control information provided by the control unit superimposed on the moving image on the viewing device. Thus, relevant control information can be provided to the surgeon directly on the viewing device.

The control unit in the instrument selection state of the graphic user interface may provide control information identifying the selected guidance instrument, and the graphic user interface may provide a marking for the display superimposed on the moving image which marks the guidance instrument in the moving image. In this way, the surgeon for example can determine which instrument tip would be centered in the middle of the image upon enabling the tracking. Thus, the marking may be used to indicate which of the instruments being used is the current guidance instrument. For example, a rectangular box or a broad line may be used as the marking which dynamically follows the movements of the guidance instrument. For example, the marking may be located above the distal shaft end of the instrument and may have a discreet, but distinctly visible color. In this way, the guidance instrument may be distinctly identifiable, without distracting the surgeon from his proper activity. On the other hand, one should avoid placing the marking directly above the instrument tip, in order for the surgeon to have a clear view of the instrument tip.

In another advantageous configuration the control unit may provide the graphic user interface with control information identifying the selected operating state, wherein the graphic user interface may provide a display of a symbol superimposed on the moving image, indicating the selected operating state in the moving image. Thus, the surgeon may obtain feedback as to the current operating state of the assistance device. For example, symbols or pictograms may be displayed at the margin of the moving image which change according to the selected operating state.

One of the operating states selectable with the switch element may be a first zoom state, in which the control unit actuates the manipulator so that an image section in the moving image is shown magnified, while another of the operating states selectable with the switch element may be a second zoom state in which the control unit actuates the manipulator so that an image section in the moving image is shown reduced.

A method for imaging support of a surgeon during a surgical procedure is also provided in the present disclosure.

### BRIEF DESCRIPTION OF THE DRAWINGS

Embodiments of the present disclosure shall now be explained more closely with the help of the following figures.

FIG. 1, a block diagram of an assistance device according to an embodiment of the present disclosure;

FIG. 2, a state diagram, showing the operating states which can be created by activating the switch element; and

FIG. 3, a table detailing the operating states provided in the sample embodiment.

### DETAILED DESCRIPTION AND INDUSTRIAL APPLICABILITY

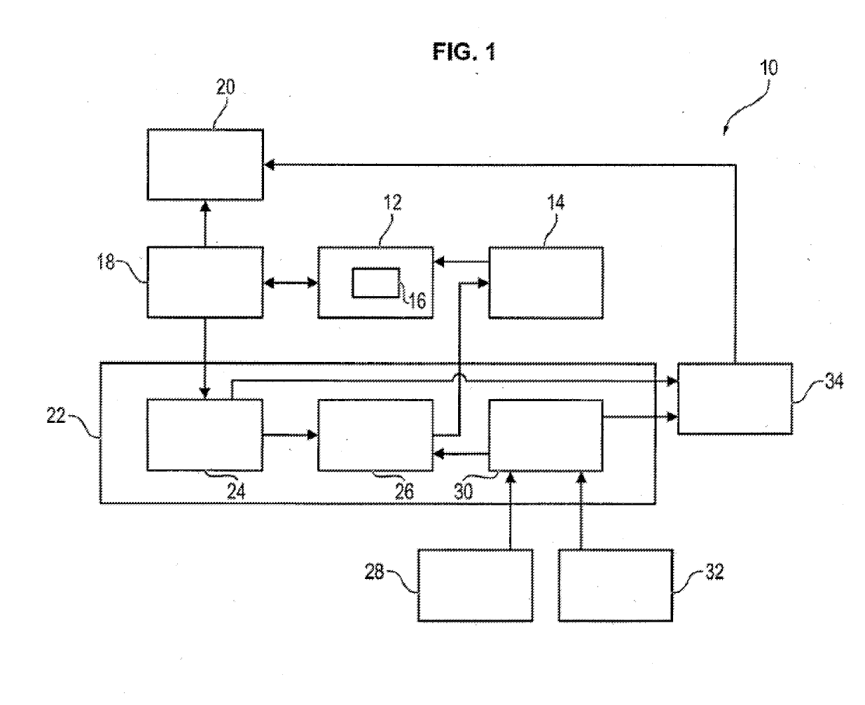

FIG. 1 shows an exemplary assistance device 10.

The assistance device 10 comprises an endoscope 12, which may be held by a manipulator 14, configured for example as a robot arm. For example, manipulator 14 may include portions of a surgical robotic system. The manipulator 14 may have mechanical degrees of freedom enabling a tracking of the endoscope 12.

The endoscope 12 may contain a camera 16, which records an image of the anatomical structure being treated. Accordingly, camera 16 may generate image data which may be put out in the form of a data stream to a camera controller 18. The camera controller 18 may relay this image data to a viewing device 20, such as a monitor screen, on which a moving image of the anatomical structure being treated may be displayed according to the image data.

The camera controller 18 may relay the image data stream via an image detection module, such as a so-called frame grabber, to a control unit 22 (e.g., a controller). The control unit 22 may contain an image processing module 24 (e.g., an image processor), which may use the image data stream supplied to it as an input signal in order to carry out an instrument recognition. The instrument recognition may occur in a known manner making use of image processing algorithms. In this process, medical instruments visible in the moving image may be recognized by the image processing module 24 and their positions may be detected. In particular, the image processing module 24 may determine a position deviation for the particular recognized instrument as to the tip of this instrument relative to the mid point of the moving image displayed on the monitor screen 20.

The image processing module 24 may put out the determined position deviations of the instrument tips to a path control 26, which may determine from this nominal values for the actuation of the manipulator 14. If appropriate, the manipulator 14 may be actuated in dependence on these nominal values to move the endoscope 12 so that the instrument tip of an instrument selected as a guidance instrument is brought to the middle of the moving image.

The assistance device 10 may have a switch element 28, which may be coupled to an interface control 30 contained in the control unit 22. The switch element 28 in the present sample embodiment may be a monostable push button, activated by pressing, with two switching states (e.g., an activated state and a non-activated state). For example, there may be two or more switching states, or there may be, e.g., precisely two switching states.

In addition to the switch element 28, which may serve as a direct control of the assistance device 10 by the surgeon, the assistance device 10 may have additional operating elements (for example, as depicted at reference numeral 32 in FIG. 1). These additional operating elements may serve to enter commands for configuring the assistance device 10 or making changes in certain control parameters, such as adjustment speeds. They can be configured in the form of a mobile entry device (e.g., with a touch sensitive surface), a remote control or an operating panel. Furthermore, they can be arranged on the manipulator 14 as entry elements (e.g., switches).

Moreover, the assistance device 10 may contain a graphic user interface 34, which may be coupled on the one hand to the image processing module 24 and interface control 30 and on the other hand to the monitor screen 20.

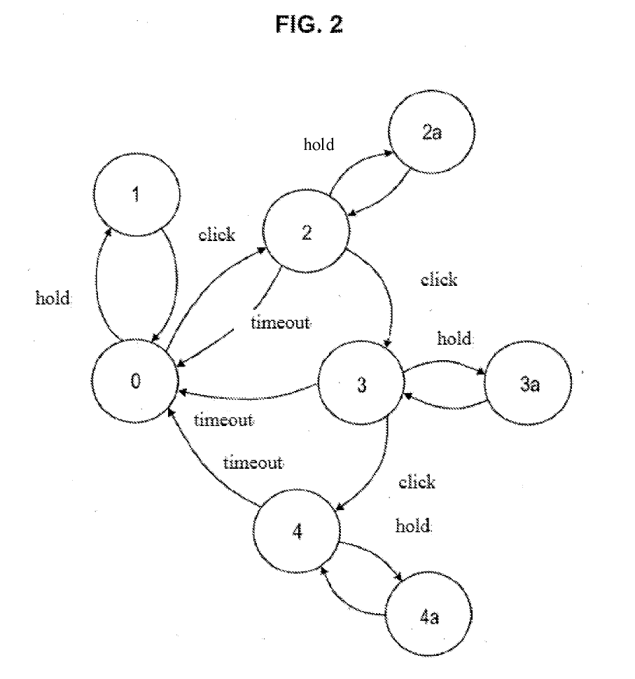

In a state diagram according to FIG. 2, for example, it is illustrated how a surgeon may use the single switch element 28 to select various operating states of the assistance device 10 and to order the control operations associated with the individual operating states. For example, the surgeon may select a current operating state from the various operating states using the single switch element 28.

As long as the surgeon does not activate the switch element 28, the assistance device 10 may be in a ground state “0”. In this ground state “0”, the path control 26 may continually compute the nominal values provided for the actuation of the manipulator 14, but as yet no actuation of the manipulator 14 with these nominal values occurs.

If the endoscope tracking is to be enabled, the surgeon may press down on the switch element 28, thereby selecting a tracking state “1”. For as long as the switch element 28 remains pressed down (“Hold” in FIG. 2), the tracking state “1” may be activated (e.g., as a current operating state or selected operating state). In the activated tracking state “1”, the endoscope tracking may be enabled, e.g., the path control 26 may actuate the manipulator 14 continually with the determined nominal values (e.g., the nominal values determined by the path control 26). Accordingly, the manipulator 14 may move the endoscope 12 so that the instrument tip is tracked. With this tracking, the instrument tip may remain in the middle of the moving image displayed on the monitor screen 20. The tracking is carried out until the surgeon releases the switch element 28 (e.g., it changes to its non-activated state). Immediately after releasing the switch element 28, the assistance device 10 may return to the ground state “0”.

If an operating state “2” is selected starting from the ground state “0”, the surgeon may perform a pulselike activation (e.g., “Click” in FIG. 2) of the switch element 28 (for example the clicking of switch element 28 when, e.g., the switching element may be for example a button or similar mechanical device as in some exemplary embodiments). Operating state “2” may be a first zoom state, in which the manipulator 14 can be actuated by a further activation of the switch element 28 so that the endoscope 12 performs a zoom movement reducing the observed image section in size. For example, this zoom movement may be performed if the surgeon once more activates and holds down the switch element 28. In this case, a control operation “2a” may be performed, coordinated with the operating state “2”, which releases the zoom movement. For as long as the switch element 28 remains held down, a continual zoom movement of the endoscope 12 may occur.

If the surgeon releases the switch element 28, the control operation “2a” may end. If the switch element 28 then remains for a particular time (such as 2 seconds) without activation, a “Timeout” (FIG. 2) may occur without any activation of the switch element 28 (e.g., the state device 10 may return to the ground state “0”).

On the other hand, if the surgeon performs a onetime pulselike activation of the switch element 28 within the aforementioned time period, the assistance device 10 may switch from operating state “2” to operating state “3”.

Operating state “3” may be a second zoom state enabling a zoom movement of the endoscope 12 by which the observed image section is enlarged. This zoom movement of the endoscope 12 may be performed if the surgeon once more activates the switch element 28 and holds it down. In this case, a control operation “3a” coordinated with the operating state “3” may be carried out, by which the zoom movement may be enabled. As long as the switch element 28 is held down, a continuous zoom movement of the endoscope 12 may occur.

If the surgeon releases the switch element and it then remains unpressed for a particular time (such as 2 seconds) (e.g., a predetermined time without pressing), the assistance device 10 may return to the ground state “0”.

On the other hand, if the surgeon performs a onetime pulselike activation of the switch element 28 within the aforementioned time period, the assistance device 10 may switch from operating state “3” to an operating state “4”.

Operating state “4” may allow for a changing of the guidance instrument displayed in the moving image and may serve as the reference for the endoscope tracking. For example, this changing of the guidance instrument may be done if the surgeon once more activates the switch element 28 and holds it down. In this case, a control operation “4a” coordinated with the operating state “4” may be carried out, which enables the changing of the guidance instrument.

While the surgeon is holding down the switch element 28, the guidance instrument visible in the moving image may be provided with a marking. After a predetermined time (such as 1 second) runs out, the marking may change to the next recognized instrument. If the surgeon would like to select a particular instrument as the guidance instrument, the surgeon may hold down the switch element 28 until the marking switches to this instrument. Once this has occurred, the surgeon may release the switch element 28. If the switch element 28 then remains for a predetermined time (such as 2 seconds) without being pressed, the assistance device 10 may automatically return to the ground state “0”. Also for example, if the surgeon performs a onetime pulselike activation of the switch element 28 within this time, the surgeon may force a switch to the ground state “0”.

As depicted in the state diagram of FIG. 2, the activations which the surgeon performs on the switch element 28 for the selection of the individual operating states may form predetermined switching patterns, which may be realized on the basis of the two switching states of the switch element 28 (for example, the activated state and the non-activated state). For example, predetermined switching patterns may be realized (e.g., solely) on the basis of two switching states of the switch element 28 (e.g., the activated state and the non-activated state). Thus, the operation of the assistance device may be easy and intuitive.

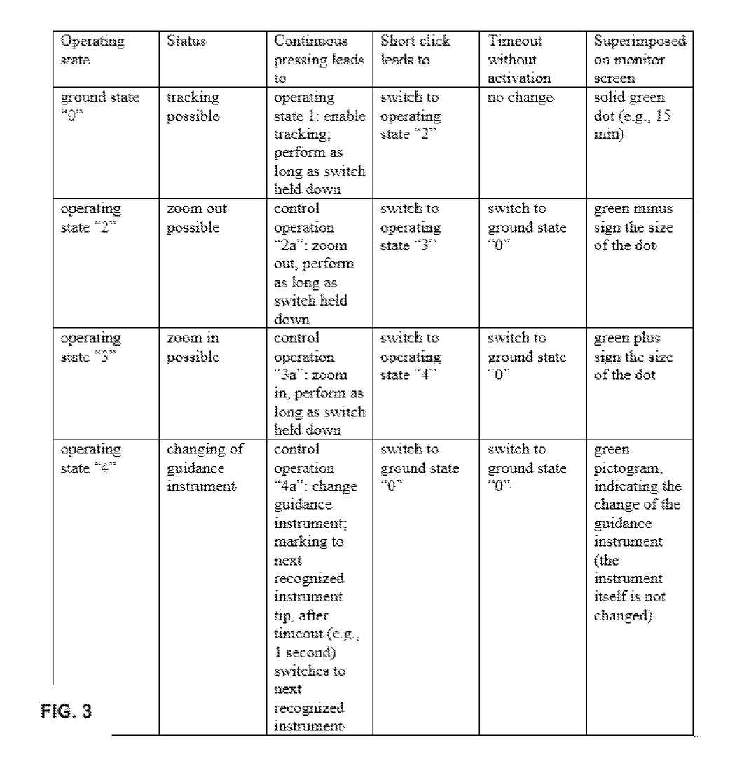

The aforementioned switching patterns for the described sample embodiments are further described in the table of FIG. 3. This furthermore shows symbols for the individual operating states which are displayed at the edge of the moving image to indicate to the surgeon, e.g., the particular operating state selected. For this, the graphic user interface 34 shown in FIG. 1 may receive information from the interface control 30 coupled to the switch element 28 as to the current switching pattern with which the switch element 28 has been activated. Moreover, the graphic user interface 34 may receive further information as to the current operating states of operating elements 32. The graphic user interface 34 may then allow for this information to be superimposed on the moving image displayed on the monitor screen 20. For example, a dot (e.g., a solid green dot or any other suitable shape and/or color for providing an indication on the viewing device 20, e.g., a dot having a diameter of between about 5 and about 30 mm, for example, of about 15 mm) may be displayed on the viewing device 20 during ground state “0,” a minus sign (e.g., a green minus sign of a size substantially equal to the above dot or any other suitable symbol for providing an indication on the viewing device 20) may be displayed on the viewing device 20 during operating state “2,” a plus sign (e.g., a green plus sign of a size substantially equal to the above dot or any other suitable symbol for providing an indication on the viewing device 20) may be displayed on the viewing device 20 during operating state “3,” and/or a pictogram (e.g., a green pictogram indicating a change of the guidance instrument, or any other suitable shape, symbol, and/or marking for providing an indication on the viewing device 20) may be displayed on the viewing device 20 during operating state “4.”

The above described embodiments are to be understood as merely exemplary. Thus, the above explained assignment of the operating states can be adapted in their sequence according to the desired attributes or characteristics set by a user.

The above-disclosed operating concept can also be expanded by additional operating states in order to, for example, activate other control operations via the switch element 28. It is also contemplated that zoom and tracking movements may be combined with each other. Thus, for example, centering the tip of the guidance instrument at the middle of the image at the same time as one of the two zoom states is enabled is also contemplated.

It will be apparent to those skilled in the art that various modifications and variations can be made to the disclosed method and apparatus. Other embodiments will be apparent to those skilled in the art from consideration of the specification and practice of the disclosed method and apparatus. It is intended that the specification and the disclosed examples be considered as exemplary only, with a true scope being indicated by the following claims.

What is claimed is:

1. A surgical operation imaging device, comprising:
an endoscope having a camera that generates image data;
a manipulator that moves the endoscope;
a viewing device that presents a moving image based on the image data;
a controller that controls the manipulator and the endoscope based on a plurality of operating states; and
an operating element coupled with the controller;
wherein the operating element has a first switching state and a second switching state;
wherein a selected operating state is selected from the plurality of operating states based on the operating element switching between the first switching state and the second switching state; and
wherein the selected operating state is changed based on the operating element switching between the first switching state and the second switching state.

2. The surgical operation imaging device of claim 1, wherein the plurality of operating states includes:
a ground state;
a first zoom state in which the moving image is shown reduced;
a second zoom state in which the moving image is shown magnified; and
a guidance instrument selection state in which a selection of a guidance instrument is selected or changed.

3. The surgical operation imaging device of claim 1, wherein the operating element is a single switch element with precisely two switching states, the precisely two switching states being the first switching state and the second switching state, with the first switching state being a non-activated state and the second switching state being an activated state.

4. The surgical operation imaging device of claim 1, wherein the operating element is switched from the first switching state to the second switching state with at least one single pulselike activation or at least one activation lasting longer than a predetermined activation time.

5. The surgical operation imaging device of claim 1, wherein:
the controller includes an image processor that detects a medical instrument in the displayed moving image based on the image data generated by the camera and ascertains a position deviation of the medical instrument relative to a reference position established within the displayed moving image; and
one of the plurality of operating states is a tracking state, in which the controller determines a nominal value based on the position deviation and actuates the manipulator based on this nominal value so that the medical instrument detected in the displayed moving image is brought into the determined reference position based on tracking of the endoscope.

6. The surgical operation imaging device of claim 5, wherein the image processor detects a tip of the medical instrument and ascertains the position deviation of the tip.

7. The surgical operation imaging device of claim 5, wherein one of the plurality of operating states is a guidance instrument selection state, in which one of a plurality of medical instruments that have been detected by the image processor in the moving image is selected as a guiding instrument, the one of the plurality of medical instruments being brought into the determined reference position in the tracking state by the tracking of the endoscope within the moving image.

8. A surgical operation imaging device, comprising:
an endoscope having a camera that generates image data;
a robot arm that moves the endoscope;
a monitor that presents a moving image based on the image data;
a controller that controls the robot arm and the endoscope based on a plurality of operating states; and
a single switch element coupled with the controller;
wherein the single switch element has a first switching state and a second switching state;
wherein a selected operating state is selected from the plurality of operating states based on the single switch element switching between the first switching state and the second switching state; and
wherein a marking is superimposed on the moving image displayed on the monitor based on the selected operating state and the single switch element switching between the first switching state and the second switching state.

9. The surgical operation imaging device of claim 8, wherein the controller includes an image processor that detects at least one medical instrument in the displayed moving image based on the image data generated by the camera and ascertains a position deviation of the at least one medical instrument relative to a reference position established within the displayed moving image.

10. The surgical operation imaging device of claim 9, wherein:
the controller provides control information identifying the at least one medical instrument as a guidance instrument; and
a graphic user interface provides the marking that is superimposed on the moving image displayed on the monitor, the marking indicating the guidance instrument.

11. The surgical operation imaging device of claim 10, wherein:
the controller provides the graphic user interface with control information identifying the selected operating state; and
the graphic user interface provides a display of a symbol superimposed on the moving image, the symbol indicating the selected operating state in the moving image.

12. The surgical operation imaging device of claim 8, wherein the single switch element is a monostable push button.

13. A method, comprising:
generating image data using a camera included in an endoscope;
displaying a moving image on a viewing device based on the image data generated by the camera;
moving the endoscope;
controlling the endoscope by using an operating element, the operating element having a non-activated state and an activated state;
changing a plurality of operating states of the endoscope by using the operating element;
switching from a ground state to a tracking state of the plurality of operating states of the endoscope by continuously activating the operating element to remain in the activated state;
returning to the ground state from the tracking state by stopping activation of the operating element to be in the non-activated state for a predetermined period of time; and
switching from the ground state to a first zoom state of the plurality of operating states of the endoscope by a single pulselike activation of the operating element that switches the operating element from the non-activated state to the activated state and then back to the non-activated state.

14. The method of claim 13, further comprising:
controlling the camera to zoom out while in the first zoom state by continuously activating the operating element to remain in the activated state; and
returning to the ground state from the first zoom state by stopping activation of the operating element to be in the non-activated state for a predetermined period of time.

15. The method of claim 14, further comprising switching from the first zoom state to a second zoom state of the plurality of operating states of the endoscope, while in the first zoom state, by a single pulselike activation of the operating element that switches the operating element from the non-activated state to the activated state and then back to the non-activated state.

16. The method of claim 15, further comprising:
controlling the camera to zoom in while in the second zoom state by continuously activating the operating element to remain in the activated state; and
returning to the ground state from the second zoom state by stopping activation of the operating element to be in the non-activated state for a predetermined period of time.

17. The method of claim 16, further comprising switching from the second zoom state to a guidance instrument selection state of the plurality of operating states of the endoscope, while in the second zoom state, by a single pulselike activation of the operating element that switches the operating element from the non-activated state to the activated state and then back to the non-activated state.

18. The method of claim 17, further comprising:
switching selection between a plurality of medical instruments displayed in the moving image while in the guidance instrument selection state by continuously activating the operating element to remain in the activated state; and
returning to the ground state from the guidance instrument selection state by either
stopping activation of the operating element to be in the non-activated state for a predetermined period of time, or
a single pulselike activation of the operating element that switches the operating element from the non-activated state to the activated state and then back to the non-activated state.

19. The method of claim 13, wherein the operating element is a push button.

20. The method of claim 19, wherein the push button has precisely two switching states, the precisely two switching states being the non-activated state and the activated state.
